(19) United States
(12) Patent Application Publication
(10) Pub. No.: US 2001/0044112 A1
(43) Pub. Date: Nov. 22, 2001

(54) High throughput screening array containing porous material

(76) Inventor: Mark B. Lyles, San Antonio, TX (US)

Correspondence Address: Baker Botts L.L.P., One Shell Plaza, 910 Louisiana, Houston, TX, 77002-4995, US

(21) Appl. No.: 09/817,009

(22) Filed: Mar. 24, 2001

Related U.S. Application Data
(60) Provisional application No. 60/192,113, filed on Mar. 24, 2000.

(51) Int. Cl. C12Q 1/68; G01N 33/53; G01N 33/537; G01N 33/543; C12M 1/34

(52) U.S. Cl. 435/6; 435/7.92; 435/287.2

(57) ABSTRACT

Two dimensional arrays of molecules on a porous material are disclosed. Combinatorial arrays of molecules may include peptides, oligosaccharides, proteins, oligonucleotides, RNA, DNA, antibodies, and small organic molecules. The arrays can be used in high throughput screening methods to identify targets, receptors, hybridizing sequences, antibody epitopes, and other chemical and biological molecule-molecule interactions.

### FIELD OF THE INVENTION

The invention relates to two dimensional arrays of molecules on porous material supports.

### BACKGROUND OF THE INVENTION

Two dimensional arrays of molecules have been suggested in the art. Glass (silica) is typically used as a support due to its ease of chemical derivatization and its relatively low price.

Arrays have been prepared using photolithography to “etch” the surface into grids, where each square of the grid contains a different chemical compound (developed and commercialized by companies such as Affymax and Affymetrix, see for example U.S. Pat. Nos. 6,040,138; 6,022,963; 5,861,242; 5,843,655; 5,831,070; and 5,744,305). This approach has been used to create two dimensional arrays of oligonucleotides. The oligonucleotide arrays have been used to detect mutations in nucleic acid sequences, and to sequence sample nucleic acid molecules. Two dimensional arrays of peptides have also been reported (Fodor, S. P. et al., Science 251(4995):767-773, 1991).

The two dimensional array concept has been referred to as a “Laboratory on a chip”, and shows considerable promise as a diagnostic tool (Talary, M. S. et al., Parasitology 117 Suppl:S191-203, 1998). Arrays of nucleic acids are commercially available from Affymetrix as a “GeneChip” (see e.g. Lipshutz, R. J. et al., Nat Genet 21(1 Suppl):20-24, 1999).

U.S. Pat. No. 5,951,295 (issued Sep. 14, 1999) describes ceramic fused fiber enhanced dental materials, and methods for their preparation. Fused-fibrous material was taught comprising from about 1% to about 50% by weight alumina, from about 50% to about 98% silica, and from about 1% to about 5% by weight boron.

U.S. Pat. No. 5,964,745 (issued Oct. 12, 1999) describes an implantable system for bone or vascular tissue. The system comprises porous linked fibrous biomaterial manufactured from nonwoven, randomly-oriented fibers linked together using a fusion source at a plurality of cross-points into a porous structure, said biomaterial having a plurality of voids of a predetermined mean void size effective for stimulating angiogenesis in said biomaterial from the tissue or bone.

U.S. Pat. No. 5,621,035 (issued Apr. 15, 1997) describes filler compositions and ceramic enhanced dental materials. The preferred embodiment of the filler composition and the ceramic dental restorative material is comprised of about 22% by weight alumina, about 78% by weight silica, about 2% by weight silicon carbide, and about 2.85% by weight boron nitride with less than 1% cristobalite contamination.

Porous materials have been suggested by Yasukawa et al. (U.S. Pat. Nos. 5,629,186 and 5,780,281). A composite was prepared from silica and/or alumina fibers with added boron nitride. The composites were suggested as being useful for cell cultures, implants, and chromatography matrices.

Conventional glass slides have less than 60% silicon dioxide on their surface. This inherently limits the amount of chemical bonding available on the surface.

There exists a need for two dimensional arrays of molecules which contain high concentrations of the molecules while maintaining the molecules in an accessible and natural conformation.

### SUMMARY OF THE INVENTION

Two dimensional arrays constructed with a porous material exhibits numerous advantages over arrays constructed with flat, nonporous materials. First, the loading capacity of the arrays is increased, leading to greater sensitivities obtainable by assays performed using the arrays. Second, due to the three dimensional properties of the porous materials themselves, chemical or biological molecules bound to the materials exhibit greater accessibility to other molecules in solution, and maintain their natural conformation better than when bound to flat, nonporous materials. Third, interactions between the bound molecules is minimized due to the three dimensional properties of the porous materials.

A wide array of chemical and biological molecules may be bound to the two dimensional array constructed with a porous material. For example, peptides, proteins, oligosaccharides, oligonucleotides, DNA, RNA, PCR products, or antibodies can be used to create the two dimensional array.

### DETAILED DESCRIPTION OF THE INVENTION

Due to the flat, two dimensional nature of the arrays, several inherent disadvantages are apparent. First, the loading capacity of the array is limited. This capacity results in lower detection capability in an assay. Second, the molecules in the array may interact with each other, or may be forced to adopt non-natural conformations due to their packing into the array. This would render them less accessible to other molecules in solution. Third, the two dimensional array would pose a steric hinderance against the binding of molecules in solution with the molecules in the array. These factors, alone or in combination, serve to reduce the efficacy of an assay using traditional two dimensional arrays of molecules.

Preparation of a two dimensional array using a porous material as a base substrate minimizes all three of these limitations. The three dimensional nature of the porous material increases loading capacity, minimizes interactions between bound molecules, improves accessibility to other molecules in solution, and helps to maintain the natural conformation of bound molecules.

Material

The porous material preferably has a three dimensional structure in order to improve accessibility of the bound compound to other compounds in solution.

The porous material can have a density of about 6 pounds per cubic foot (96.1 kg/m³) and higher, about 8 pounds per cubic foot (128 kg/m³) and higher, about 12 pounds per cubic foot (192 kg/m³) and higher, about 24 pounds per cubic foot (384 kg/m³) and higher, about 36 pounds per cubic foot (577 kg/m³) and higher, about 48 pounds per cubic foot (769 kg/m³) and higher, or about 64 pounds per cubic foot (1025 kg/m³) and higher. The material can comprise up to about 100% silica, or up to about 60% alumina. The silica may be up to about 50% cristobalite, up to about 75% cristobalite, up to about 90% cristobalite, up to about 95% cristobalite, up to about 99% cristobalite, or can be about 100% cristobalite. The alumina can be aluminum borosilicate.

The exposed surface of the material (“surface chemistry”) can be at least about 50% silicon dioxide, at least about 75% silicon dioxide, at least about 90% silicon dioxide, at least about 95% silicon dioxide, at least about 99% silicon dioxide, or can be about 100% silicon dioxide.

The material can comprise other metal oxides in addition to or in place of the silica. For example, tantalum oxide or zirconium oxide can be incorporated into the material. The material can be reinforced with silica gel.

Laser etching can be used to etch holes in the material. Hydrofluoric acid etching can be performed to increase surface area of the material.

The mean pore diameter of the material can be less than 0.01 microns, about 0.1 micron to about 5 microns, up to about 10 microns, up to about 20 microns, up to about 30 microns, up to about 40 microns, up to about 50 microns, up to about 100 microns, up to about 200 microns, up to about 300 microns, up to about 400 microns, up to about 500 microns, or up to about 600 microns. Ranges of pore diameter include about 0.1 microns to about 1 micron, about 5 microns to about 10 microns, about 20 microns to about 50 microns, about 100 to about 400 microns, or about 200 microns to about 600 microns.

The surface properties of the material can be modified by chemical reactions. Examples include modifying the hydrophobicity or hydrophilicity of the porous materials, hydroxylation with phosphoric acid, and activation with molecular oxygen.

The following method of preparing a porous material was described in U.S. Pat. No. 5,629,186.

Preparing the Matrix

In general, the method includes forming a fiber slurry having desired viscosity and fiber dispersion characteristics, allowing the slurry to settle under conditions that produce a selected fiber density and orientation, drying the resulting fiber block, and sintering the block to form the desired fused-fiber matrix.

A. Fiber Treatment

The silica (SiO₂) and/or alumina (Ale₂O₃) fibers used in preparing the matrix are available from a number of commercial sources, in selected diameters (fiber thicknesses) between about 0.5 μm-20 μm. A preferred silica fiber is a high purity, amorphous silica fiber (99.7% pure), such as fabricated by Manville Corporation (Denver, Col.) and sold under the fiber designation of “Q-fiber”. High purity alumina fibers (average 3 microns) may be procured, for example, from ICI Americas, Inc. (Wilmington, Del).

In a preferred heat treatment, the silica fibers are compressed into panels, e.g., using a Torit Exhaust System and compaction unit. The compressed panels are sent passed through a furnace, e.g., a Harper Fuzzbelt furnace or equivalent at 220° F. for 120 minutes, corresponding to a speed setting of about 2.7 inches/minute. The heat treatment is used to close up surface imperfections on the fiber surfaces, making the matrix more stable to thermal changes on sintering. The heat treatment also improves fiber chopping properties, reducing fabrication time.

In a preferred method, the heat-treated fibers are washed to remove debris and loose particles formed during fiber manufacturing.

B. Preparing a Fiber Slurry

Silica and/or alumina fibers from above are blended to form a fiber slurry that is used in forming a “green-state” block that can be sintered to form the desired matrix.

The slurry is formed to contain, in an aqueous medium, silica, alumina, or silica and alumina fibers of the type described above, at a fiber:liquid weight ratio of between about 1:25 to 1:70, where the liquid weight refers to the liquid weight of the final slurry preparation.

The slurry preferably includes a thickening agent effective to give the slurry a viscosity between about 1,000 and 25,000 centipoise, as measured by standard methods. The viscosity agent may be any of a number of well-known hydrophilic polymers, such as polyvinylalcohol, polyvinylacetate, polyvinylpyrrolidone, polyurethane, polyacrylamide, food thickeners, such as gum arabic, acacia, and guar gum, and methacrylate type polymers. The polymers preferably have molecular weights greater than about 25-50 Kdaltons, and are effective to increase solution viscosity significantly at concentrations typically between about 0.5-10 weight percent solution.

Preferred thickening agents polymers that also have tacky or adhesive properties, such as methyl cellulose, terpolymers of maleic anhydride, alkyl vinyl ether, and an olefin (U.S. Pat. No. 5,034486), copolymers of ethylene and olefins (U.S. Pat. No. 4,840,739), cellulose-containing pastes (U.S. Pat. No. 4,764,548), and soy polysaccharides. One preferred thickening agent is methylcellulose, e.g., the polymer sold under the tradename Methocel A4M and available from Dow Chemical Co. (Midland, Mich). Where the matrix is formed of high-purity silica fibers and/or alumina, the slurry is also formed to contain a source of boron that functions, during sintering, to form a boron/silica or boron/alumina surface eutectic that acts to lower the melting temperature of the fibers, at their surfaces, to promote fiber/fiber fusion at the fiber intersections. In a preferred embodiment, the boron is supplied in the slurry as boron nitride particles 15 to 60 μm in size particles. Such particles can be obtained from Carborundum (Amherst, N.Y.). The amount of boron nitride is preferably present in the slurry in an amount constituting between about 2-12 weight percent of the total fiber weight.

The adhesive property of the thickening agent described above is useful in adhering particles of boron nitride and, if used, silicon carbide, to the fibers in the slurry, to produce a relatively uniform of particles in the slurry, and prevent the particles from settling out of slurry during the molding process described below.

The slurry preferably also contains a dispersant which acts to coat the fibers and help disperse the fibers, both to increase slurry viscosity, and to prevent silica fibers from “bundling” and settling out of the slurry as fiber aggregates during the molding process described below. The dispersant is preferably one which imparts a significant charge and/or hydrophilicity to the fibers, to keep the fibers separated during slurry formation and settling during the molding process.

For use with silica fibers, ammonium salts are particularly useful as dispersants, because the ammonium cation is released from the matrix in the form of ammonia during matrix drying and/or sintering. Preferred ammonium salts are the salts of polyanionic polymers, such as ammonium polymethylmethacrylate, or the ammonium salt of other carboxylated polymers. One preferred dispersant agent is the ammonium polymethylmethacrylate polymer sold by R. T. Vanderbilt under the tradename Darvan 821 A. The polymer dispersant is preferably added to the slurry to make up between about 0.2 to 5 percent of the total liquid volume of the slurry.

The slurry may further contain between about 1-5 percent by weight silicon carbide particles, such as obtainable from Washington Mills Electro Minerals Corp. (Niagara Fall, N.Y.).

A preferred method for preparing a slurry of the type just described is detailed as follows. Briefly, heat-treated silica fibers are suspended in water at a preferred fiber:water ratio of about 1:300 to 1:800. The fiber slurry is pumped through a centrifugal cyclone to remove shot glass and other contaminants, such as high soda particles. The fiber cake formed by centrifugation is cut into segments, dried at 550° F for at least 8 hours, and then broken into smaller chunks for forming the matrix.

Fragments of the silica fiber cake are mixed in a desired weight ratio with alumina fibers, and the fibers are dispersed in an aqueous solution containing the dispersing agent. At this point, the fibers are preferably chopped to a desired average fiber length in a lowshear/high-shear mixer. In general, the greater the degree of chopping, the shorter the fibers, producing better packing and a less open matrix structure. Similarly, longer fibers lead to more open matrix structure. The fiber mixing is preferably carried out under condition to produce average fiber sizes of a selected size in the 1-10 mm fiber-length range.

After mixing, the fibers are allowed to settle, and the liquid/fiber ratio is reduced by decanting off some of the dispersing liquid. To this slurry is added an aqueous gel mixture formed of the viscosity agent, e.g., methyl cellulose, and the matrix particles, e.g., boron nitride particles, and the slurry components are mixed to form the desired high-viscosity slurry. The slurry is now ready to be transferred to a casting mold, to prepare the green-state block, as described in the next section.

C. Forming a Dried Fiber Block

According to an important aspect of the method, the slurry is allowed to settle and is dewatered in a fashion designed to achieve a relatively uniform fiber density throughout the matrix, and relatively randomly oriented fibers, i.e., with little a fiber orientation preference in the direction of settling.

In the first step, a slurry is added to a mold equipped with a lower screen sized to retain slurry fibers. For fiber sizes in the range 1-10 mm, the screen has a mesh size between about 8 to 20 squares/inch. The mold has a lower collection trough equipped with a drain and a vacuum port connected to a suitable vacuum source.

Initially, the slurry is added to the mold and, after stirring the slurry to release gas bubbles, is allowed to settled under gravity, until the level of water in the mold is about 1-2 inches above the level of the desired final compaction height, i.e., the final height of the dewatered block. For a slurry of about 12-1 added to a 18 cm² square mold, the initial settling takes about 3-10 minutes.

The partially drained slurry in the mold is now compacted with a compacting ram to force additional water from slurry. This is done by allowing the ram to act against the upper surface of the slurry under the force of gravity, while draining the water forced through a screen from the mold. Water is squeezed from the slurry until the ram reaches the desired compaction height. With the slurry volume and mold dimensions just given, a ram having a weight of about 7 lbs is effective to compress the partially dewatered slurry in a period of about 0.2 to 2 minutes.

In the final step of compacting and dewatering, the drain is closed and vacuum is applied to a port until the block is completely dewatered. A vacuum of between about 0.01 to 0.5 atm is effective to produce complete dewatering of the mold in a period of about 0.2 to 5 minutes. The vacuum dewatering may result in the upper surface of the block pulling away from the ram.

The dewatered block is now removed from the mold and dried in an oven, typically at a temperature between 250° F.-500° F. In the dried matrix, the viscosity agent, and to a lesser extent, the dispersant agent, act to bond the fibers at their intersections, forming a rigid, non-fused block. The target density of the matrix after drying is between about 3.3 to 5.3 pounds/ft³.

The green-state matrix may be formed to include sacrificial filler element(s) that will be vaporized during sintering, leaving desired voids in the final fused matrix block. The filler elements are preferably formed of polymer or graphite. An array of parallel rods may be placed in the mold, at the time the slurry is added. Slurry settling and dewatering are as described above, to form the desired green-state block with the included sacrificial element.

The first step is the slurry formation. The slurry may be a single fiber suspension containing a desired size range and fiber composition. Alternatively, for forming a discontinuous or step fiber matrix, two or more slurries having different fiber thicknesses, densities, and/or fiber compositions may be formed.

After the slurry is introduced into the mold, the steps in settling and dewatering the slurry can be varied to produce either a continuous gradient of fiber density or a uniform fiber density. The steps in forming a uniform gradient, including an initial settling step, followed by ram compaction and final dewatering by vacuum have been considered above.

To produce a continuous gradient of fiber densities, the slurry is first subjected by dewatering by vacuum, causing material closest to the screen to be compacted preferentially. When a desired gradient is achieved, the slurry is gravity drained to dewater the slurry, then ram-compacted for further dewatering. The slurry may be subjected to a final vacuum dewatering.

To produce a block having a series of discontinuous layers, each with a uniform fiber density, each successive slurry is handled substantially as described above for the uniform-density block. The layers can be formed by successively casting layer upon layer in the mold, with each successive layer being compacted as described above.

Alternatively, a series of block layers, each with a distinctive fiber size/composition and/or density is prepared. Before drying, the individual blocks are placed together in is layers, to form the desired discontinuous-layer block. The layers may be “glued” together before drying by applying, for example, a layer of boron nitride in the viscosity agent between the layers.

D. Fused Fiber Matrix

In the final step of matrix formation, the green-state block from above is sintered under conditions effective to produce surface melting and fiber/fiber fusion at the fiber crossings. The sintering is carried out typically by placing the green-state block on a prewarmed kiln car. The matrix is then heated to progressively higher temperature, typically reaching at least 2,000° F., and preferably between about 2,200° F.-2,400° F., until a desired fusion and density are achieved, the target density being between 3.5 and 5.5 pounds/ft³. For a block formed solely of alumina fibers, a maximum temperature of about 2,350° F. is suitable.

In a preferred method, discussed above, the matrix is formed with high-purity silica fibers that contain little or no contaminating boron and/or with alumina fibers that are also substantially free of boron. In order to achieve fiber softening and fusion above 2,000° F., typically in the temperature range 2,000° F.-2,200° F., it is necessary to introduce boron into the matrix during the sintering process, to form a silica/boron or alumina/boron eutectic mixture at the fiber surface. Boron is preferably introduced, as detailed above, by including boron nitride particles in the green-state block, where the particles are evenly distributed through the block.

During sintering, the boron particles are converted to gaseous N₂ and boron, with the released boron diffusing into the surface of the heated fibers to produce the desired surface eutectic, and fiber fusion. The distribution of boron particles within the heated block ensures a relatively uniform concentration of boron throughout the matrix, and thus uniform fusion properties throughout.

Also during fusion, the viscosity agent and dispersant agents used in preparing the green-state block are combusted and driven from the block, leaving only the fiber components, and, if added, silicon carbide particles.

Where the green-state block has been constructed to include a sacrificial element, the sintering is also effective to vaporize this element, leaving desired voids in the matrix, such as a lattice of channels throughout the block.

After formation of the fused-fiber matrix, the matrix block may be machined to produce the desired shape and configuration. For example, the matrix can be formed by drilling an array of channels in the block; or by cutting the block into thin plates.

Polymer Fiber Matrix

In another aspect, the invention includes a fibrous polymer matrix. The matrix is composed of fused polymer fibers, and is characterized, in dry form, by: (a) a rigid, three-dimensionally continuous network of open, intercommunicating voids, and (b) a free volume of between about 90-98 volume percent. The fibers may also include up to 80 percent by weight of either silica fibers, alumina fibers, or a combination of the two fibers types.

The matrix is designed for use particularly as a substrate for cell growth in vitro, and as such, contains an array of channels extending through the matrix. In an alternative embodiment, the matrix has a multi-plate configuration.

The fused polymer matrix is formed substantially as described for the silica, alumina, or silica/alumina fiber matrices described above, but with the modifications now to be discussed.

The polymer fibers used in constructing the matrix may be any thermoplastic polymers that can be heat fused, typically when heated in the range 400° F.-800° F. Exemplary polymer fibers include polyimide, polyurethane, polyethylene, polypropylene, polyether urethane, polyacrylate, polysulfone, polypropylene, polyetheretherketone, polyethyleneterphthalate, polystyrene, and polymer coated carbon fibers. Fibers formed of these polymers, and preferably having thickness in the 0.5 to 20 μm range, can be obtained from commercial sources. The fibers may be chopped, i.e., by shearing, to desired lengths, e.g., in the 0.1 to 2 mm range, by subjecting a suspension of the fibers to shear in a high-shear blender, as described above.

The polymer fibers may be blended with up to 80 weight percent silica and/or alumina fibers of the type described above. Preferably, the silica fibers are heat treated to close up surface imperfections on the fiber surfaces, as described above. The alumina fibers may also be heat treated, e.g., under the sintering conditions described above, to produce surface granulation on the fiber.

The aqueous fiber slurry used in preparing the matrix contains, in addition to fibers, a viscosity agent effective to produce a final slurry viscosity between about 1,000 and 25,000 centipoise. Viscosity agents of the type mentioned above are suitable. If the polymers fibers are relatively hydrophobic, or if the fibers include silica fibers, the slurry should contain a dispersant effective to prevent the fibers from aggregating on settling. Such a dispersant may include surfactants and/or charged polymers, and/or block copolymers, such as polyethylene/polypropylene block copolymers known to enhance the hydrophilicity of polymer surfaces.

The slurry also contains an adhesive agent effect to retain the green-state fiber network in a rigid condition once it is formed. Either the viscosity agent or dispersant may supply the necessary adhesive properties. Alternatively, a separate adhesive component may be added to the slurry.

The above slurry is placed in a settling mold, as above, and the fibers are allowed to settle under dewatering conditions, substantially as described above, to yield randomly oriented fibers having a desired fiber density. The network is formed into a greenstate block by drying, e.g., at 100° F.-300° F.

In the final step, the greenstate block is heated under conditions, typically at a temperature between 400° F.-800° F., effective to produce fiber fusion at the fiber points of intersection. The selected temperature is near the softening point of the thermoplastic polymer. At this temperature, the polymer fibers fuse with one another and with silica and/or alumina fibers in the block to produce the desired rigid, fused fiber matrix.

Arrays

The two dimensional array can be constructed entirely of the porous material, or can comprise a layer of porous material mounted on top of a flat surface such as glass, plastic, or metal. The porous material can be adhered to the flat surface by an adhesive, applied using a solvent, or cast directly onto the flat surface.

The two dimensional array can be formed on a strip of material. For example, a strip of at least about 300 microns in thickness can contain miniwells formed by laser etching. Alternatively, a strip can have bands of bound molecules.

The two dimensional arrays can generally be constructed with any type of bound molecule. The two dimensional arrays are particularly attractive in the assay of combinatorial libraries of molecules, where the binding of a wide array of molecules to a given receptor or target may be investigated quickly and efficiently. Examples of molecules useful for analysis with two dimensional arrays include peptides, proteins, oligosaccharides, oligonucleotides, DNA, RNA, PCR products, and antibodies. Traditional organic chemistry has been increasingly being performed on two dimensional arrays to construct combinatorial libraries of drugs and other molecules of interest.

The array of molecules bound to the porous material is preferably in a grid pattern. The number of different molecules in the array is preferably at least about 100, more preferably at least about 100, 250, 500, 750, 1000, 2000, 3000, 4000, 5000, 6000, 7000, 8000, 9000, 10000, 50000, 100000, 250000, 500000, or 1000000. The number of different molecules may be increased by either increasing the physical size of the two dimensional array in order to allow for a greater number of grid positions, and/or by decreasing the size of the grid positions in order to allow for a greater number of grid positions per square area.

The two dimensional arrays of molecules bound on a porous material can be used in the detection of a target molecule in a sample. A method for the detection of a target molecule in a sample preferably comprises obtaining a two dimensional array of molecules bound on a porous material, wherein one or more of the molecules is a partner molecule which binds to the target molecule; contacting the two dimensional array and the sample to produce a partner molecule-target molecule complex; and detecting the location of the partner molecule-target molecule complex in the two dimensional array. The location of the complex in the two dimensional array permits identification of the identity of the partner molecule which bound to the target molecule. The partner molecule may be covalently or non-covalently bound to the porous material. The partner molecule may generally be any type of molecule, and preferably is a peptide, an oligosaccharide, a protein, an oligonucleotide, or an antibody. The partner molecule-target molecule complex may be detected by any method, and preferably is detected by fluorescence, radioactivity, ultraviolet spectroscopy, or visible spectroscopy. Visible spectroscopy may include color changes or light emission. The detection step may be qualitative or quantitative. The method may be performed manually or by automated is robotics, with the use of automated robotics being preferred.

Preparation of porous materials

The preparation of porous materials is generally described in U.S. Pat. No. 5,951,295 (issued Sep. 14, 1999).

Porous materials can be prepared from: (1) from about 1% to about 50% by weight alumina; (2) from about 50% to about 98% by weight silica; and (3) from about 1% to about 5% by weight boron. In addition, the composition may further comprise silicon carbide up to about 3% by weight. The materials can comprise over 99% silica.

Generally, the process for preparing the porous materials can comprise the following steps (as described in U.S. Pat. No. 5,951,295):

(1) preparation of a slurry mixture comprised of pre-measured amounts of purified fibers/materials and deionized water;

(2) removal of shot from slurry mixture;

(3) removal of water after thorough mixing to form a soft billet;

(4) addition of a ceramic binder after the formation of the billet;

(5) placement of the billet in a drying microwave oven for moisture removal;

and

(6) sintering the dry billet in a large furnace at about 1600° F. or above.

The high purity silica fibers above are first washed and dispersed in hydrochloric acid and/or deionized water or other solvents. The ratio of washing solution to fiber is between 30 to 150 parts liquid (pH 3 to 4) to 1 part fiber. Washing for 2 to 4 hours generally removes the surface chemical contamination and non-fibrous material (shot) which contributes to silica fiber devitrification. After washing, the fibers are rinsed 3 times at approximately the same liquid to fiber ratio for 10 to 15 minutes with deionized water. The pH is then about 6. Excess water is drained off leaving a ratio of 5 to 10 parts water to 1 part fiber. During this wash and all following procedures, great care must be taken to avoid contaminating the silica fibers. The use of polyethylene or stainless steel utensils and deionized water aids in avoiding such contamination. The washing procedure has little effect on the bulk chemical composition of the fiber. Its major function is the conditioning and dispersing of the silica fibers.

The alumina fibers are prepared by dispersing them in deionized water. They can be dispersed by mixing 10 to 40 parts water with 1 part fiber in a V-blender for 21/2 to 5 minutes. The time required is a function of the fiber length and diameter. In general, the larger the fiber, the more time required.

Generally, in order to manufacture low density porous materials, for example, densities below 12 lb/ft³ ((192 kg/m³)), the process includes the additional steps of:

(1) the addition of expendable carbon fibers in the casting process and/or other temporary support material; and

(2) firing the billet at about 1300° F. to remove the carbon fibers or other support material prior to the final firing at approximately 1600° F. or above.

When the dispersed silica fibers and dispersed alumina fibers are combined, the pH may be acidic, and if so, should be adjusted to neutral with ammonium hydroxide. The slurry should contain about 12 to about 25 parts water to about 1 part fiber. The slurry is mixed to a uniform consistency in a V-blender in 5 to 20 minutes. The boron nitride can be added at this point (2.85% by weight of the fibers) and mixed to a uniform consistency in a V-blender for an additional 5 to 15 minutes creating a Master Slurry. The preferred mixing procedure uses 15 parts water to 1 part fiber and the slurry is produced in about 20 minutes of mixing. At lower density formulations, expendable carbon fibers are used to give “green” strength to the billet prior to the final sintering. The percent of carbon fiber used varies greatly depending on the diameter, length and source of the fiber and the ultimate density of the material being produced. The percent of carbon fiber per dry weight of material should range between 1% and 10%. The source of the carbon fiber can take many forms including nylon, cellulose, and purified graphite based carbon in fibrous form. Carbon fibers added in the casting process are eliminated by firing the billets at 1350° F. prior to the final firing at 2450° F.

The Master Slurry is poured into a mold for pressing into the desired shape. The water is withdrawn rapidly and the resulting felt is compressed at 10 to 20 psi. Rapid removal of the water is required to prevent the fibers from separating. If graded properties are desired in the resultant material, the slurry can be allowed to settle and the fibers to partially separate before the removal of the water.

The final density of the finished restorative material is determined in part by the amount of compression placed on the felt, varying the wet molded dimension in relation to the fiber content. The formulation of the present invention has been prepared in densities ranging from about 0.05 to 0.48 g/cc. It can, however, be prepared in lower and higher densities.

After molding, the restorative material can be dried and fired by the following procedure. The material is first dried in an oven for 18 hours; the temperature, initially 38° C, is raised at a rate of 11° C. per hour to 104° C., held there for 4 hours, raised again at a rate of 11° C. per hour to 150° C., and held there for 4 hours. The material is taken directly from the drying oven, placed in the firing furnace, and fired. A temperature rise rate of 220° C. per hour or less is required in order to avoid cracking and warping in the case of a 15 cm×15 cm×7.5 cm block of material. For larger blocks, slower heating rates may be required. The maximum firing temperature may vary from 1200° C. to 1600 ° C. depending upon the fiber ratio used, amount of boron nitride, and the final density of the material that is desired.

The temperature rise rate is chosen to permit relatively uniform temperatures to be achieved throughout the material during the process. A faster temperature rise rate causes non-uniform temperatures to be achieved throughout the material during the process. A faster temperature rise rate causes nonuniform strength and density and may cause cracking. Longer or higher temperature firing results in higher shrinkage and related greater resistance to subsequent shrinkage, as well as a shorter lifetime to devitrification under cyclic exposures to high temperatures. The maximum firing temperature is dependent upon the fiber ratio used and the density of the composite desired. The firing time and maximum temperature are selected to allow sufficient shrinkage to achieve stabilization and fiber fusion while not allowing any devitrification. After firing, the material may be machined to obtain any desired final dimensions.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention.

What is claimed is:

1. A two dimensional array comprising molecules bound to a porous material surface; wherein:
the array contains at least about 100 different molecules; and
each of the different molecules is bound in a different predetermined region of the surface.

2. The array of claim 1, wherein the porous material comprises alumina and silica.

3. The array of claim 1, wherein the porous material is made from alumina, silica, and boron.

4. The array of claim 1, wherein the porous material is made from a composition comprising about 1% to about 50% by weight alumina, about 50% to about 98% by weight silica, and about 1% to about 5% by weight boron.

5. The array of claim 1, wherein the mean pore diameter of the porous material is greater than about 10 microns.

6. The array of claim 1, wherein the density of the porous material is at least about 6 pounds per cubic foot (96.1 kg/m³).

7. The array of claim 1, wherein the exposed surface is at least about 50% silicon dioxide.

8. The array of claim 1, wherein the exposed surface is at least about 75% silicon dioxide.

9. The array of claim 1, wherein the exposed surface is at least about 95% silicon dioxide.

10. The array of claim 1, wherein the molecules are oligonucleotides.

11. The array of claim 1, wherein the molecules are peptides.

12. The array of claim 1, wherein the molecules are oligosaccharides.

13. The array of claim 1, wherein the molecules are DNA.

14. The array of claim 1, wherein the molecules are RNA.

15. The array of claim 1, wherein the molecules are proteins.

16. The array of claim 1, wherein the molecules are antibodies.

17. A method for the detection of a target molecule in a sample, the method comprising:
obtaining a two dimensional array comprising molecules bound to a porous material surface, wherein:
the array contains at least about 100 different molecules;
at least one of the different molecules is a partner molecule which binds to the target molecule; and
each of the different molecules is bound in a different predetermined region of the surface;
contacting the array and the sample to produce a partner molecule-target molecule complex; and
detecting the partner molecule-target molecule complex.

18. The method of claim 17, wherein the partner molecule is covalently bound to the porous material.

19. The method of claim 17, wherein the porous material comprises alumina and silica.

20. The method of claim 17, wherein the porous material is made from a composition comprising alumina, silica, and boron.

21. The method of claim 17, wherein the porous material is made from a composition comprising about 1% to about 50% by weight alumina, about 50% to about 98% by weight silica, and about 1% to about 5% by weight boron.

22. The method of claim 17, wherein the mean pore diameter of the porous material is greater than about 10 microns.

23. The method of claim 17, wherein the exposed surface is at least about 50% silicon dioxide.

24. The method of claim 17, wherein the exposed surface is at least about 75% silicon dioxide.

25. The method of claim 17, wherein the exposed surface is at least about 95% silicon dioxide.

26. The method of claim 17, wherein the partner molecule is a peptide.

27. The method of claim 17, wherein the partner molecule is an oligosaccharide.

28. The method of claim 17, wherein the partner molecule is a protein.

29. The method of claim 17, wherein the partner molecule is an antibody.

30. The method of claim 17, wherein the partner molecule is an oligonucleotide.

31. The method of claim 17, wherein the partner molecule is DNA.

32. The method of claim 17, wherein the partner molecule is RNA.

33. The method of claim 17, wherein the partner molecule-target molecule complex is detected by fluorescence.

34. The method of claim 17, wherein the partner molecule-target molecule complex is detected by radioactivity.

35. The method of claim 17, wherein the partner molecule-target molecule complex is detected by visible spectroscopy.

36. The method of claim 17, wherein the partner molecule-target molecule complex is detected by ultraviolet spectroscopy.
